(19) United States
(12) Patent Application Publication
(10) Pub. No.: US 2003/0220590 A1
(43) Pub. Date: Nov. 27, 2003

(54) Electro-mechanical assembly for measurements of spine curvatures

(76) Inventor: Paul Janos Csonka, Eugene, OR (US)

Correspondence Address: Paul J. Csonra, 105 E 39th Avenue, Eugene, OR, 97405, US

(21) Appl. No.: 10/151,760

(22) Filed: May 22, 2002

(51) Int. Cl. A61B 5/103

(52) U.S. Cl. 600/594

(57) ABSTRACT

The Electro-Mechanical Assembly for Measurements of Spine Curvatures is an inexpensive, accurate, and efficient means of measuring spine position (flexation), along its entire length, and in dynamic and remote test situations.

In one embodiment of the invention, sliding contact rods partially protrude from the rear surface of a standard modified host backpack, with the rods generally perpendicular to the surface of the human test-subject's back. These contact rods mayy be spring-mounted, so as to remain in contact with the spine, even during spine flexation. Linear sensing contact rod sensors can sense the movement of a given sliding contact rod; this movement is directly coupled with the spine segment position the contact rod is touching. Thus, the positions of the contact rod sensors are a direct translation of the spine position. A micro-controller on-board can read the sensor positions, and all such data are recorded on-board, to be downloaded to a host computer for analysis.

### BACKGROUND OF THE INVENTION

1. Field of the Invention

This invention relates to the general field of research in the medical community, primarily physical therapy fields, and is also of interest to backpack manufacturers and designers, in particular the armed forces' research and development branches.

2. Description of Prior Art

There are no mobile spine-measuring devices to date, which allow provide optional data-logging abilities. However, interest has been expressed for a device that can detect back posture easily and accurately, and also the underlying spine position for analysis, and this invention is a result of such interest.

There is a lack of complete understanding on how the spine behaves under various load carriages, such as prolonged backpack use. In particular, there is concern about the health of school-going children, where backpacks are a common means of transporting heavy textbooks and other materials. In addition, the various military branches also have an interest in obtaining data on how exactly the spine behaves while under the substantial backpack loads of an infantry soldier, as minimizing discomfort, bad posture, and fatigue maximizes a soldier's health, productivity, and efficiency.

Improving backpack designs to minimize back pain and damage—and maximize comfort, health, carrying ability, and safety—is an ongoing effort. But these efforts are thwarted by the lack of devices for actually measuring spine shape, easily and with low cost, during dynamic test situations. This invention fills that void.

### BRIEF SUMMARY OF THE INVENTION

The invention described here is a simple and inexpensive device suitable for obtaining real-time data of spine activity, both independently, and under the load of a backpack in an alternate embodiment of the invention, while the test-subject is either at rest or moving. These situations include, but are not limited to, sitting, walking, crawling, and climbing.

This invention measures spine position and contours along the entire length of the spine, with a resolution desired by the user, to a maximum resolution set by the geometric nature of the invention. To measure spine position, many spring-mounted non-flexible rods extend perpendicularly from the backpack surface nearest the spine, and move in and out in accordance to the given spine segment position. The measurement of these rod positions directly characterizes the position of the spine.

In an embodiment where this invention is set to record the sliding contact rod positions using a recording device carried by the test-subject, the invention has an on-board microprocessor, which records rod position data into memory at any set time(s), and in any pattern set by the user. This allows remote spine measuring, and frees the test-subject from being connected to a computer.

An advantage of this invention is that data analysis is inherently straightforward and simple, due to the linear coupling of spine position to sensor output, even if the sensor outputs themselves are not linear with the rod positions. However, inexpensive linear output vs. position sensors are easily obtained, such as the linear-taper linear-slide variable resistors, or Linear Variable Differential Transformers, which can use the rod directly as the sensing element.

The per-use and total lifetime operation of this invention depends on the selection of components. The two most crucial ones include batteries for peruse operation of the processor and sensors, and for operational lifetime, the operational lifespan of the sensors themselves. Because microprocessors have low power consumption, and the invention is designed to measure backpack toads, heavy high-capacity batteries, to help simulate loads, are generally acceptable even if they are not necessary.

Due to the inexpensive nature of even high-cycle-life linear variable resistors, and the wide assortment of types, manufacturers, and mounting options of these resistors, the variable resistors may be easily and quickly replaced in the invention should the need arise.

Therefore, the operational cost of the invention arises mainly from the replacing of batteries. The use of rechargeable battery technology virtually eliminates the battery replacement cost.

Because of the flexible nature of micro-controllers, many data-collection schemes can be configured, enabling the user to collect data in a manner specific to their application. The invention can be used both in an embodiment of a data-logging device, where the on-board micro-processor stores all position data, or in a tethered embodiment, where data-collection is limited only by the amount of memory on the connected host desktop computer, and movement of the test-subject limited only by the length of the computer cable connecting the invention to the said host computer. Alternately, some data may be transmitted to a host computer, and other data stored on-board.

For more flexibility, aside from pre-programmed data collection, an external trigger (such as a Radio Frequency or infra-red remote control) may trigger the recording of data in the backpack. This may occur in situations where the invention is used in data-logging mode, and the user does not desire a set recording sequence, and the invention does not have enough memory to continuously record data for an entire trial run, which may be set to last several hours. In this situation, an option is to remotely trigger recording of intervals where the person administering the test deems that the collection of data would be advantageous.

The nature of the error in trigonometric functions (small error at small angles) makes the invention robust in terms of accuracy of the translation of spine position to rod position. In the case that the invention is used in the embodiment requiring a modified host backpack, and the invention is used without elastic waist-support straps, there is a potential for the backpack to swing away from the test subject's back. But small angle deflections cause minimal and acceptable errors in the positions of the perpendicular sliding measurement rods.

Any backpack being tested must be minimally and easily, but irreversibly, modified to create this embodiment of the invention, as specified in the drawings. The advantage is that the user can select the specific production backpack's, or pre-production prototype's, whose properties are to be tested, allowing rapid design development.

This invention allows multiple rows of sensor rods to be placed in a grid-like pattern, instead of a single row that exclusively measures the spine, so that the position of all parts of the test subject's back can be measured. In this configuration, special attention must be given to ensure that all rods move in a low-friction environment, in order that the return springs can have a lower spring constant (k) value, and thus the total pressure on the test-subject's back be minimized, causing less deflection of the device away from the human back.

Further advantages of the invention and its simplicity will become apparent upon examination of the ensuing summary, drawings, and detailed description of the invention.

### BRIEF DESCRIPTION OF THE SEVERAL VIEWS OF THE DRAWING

FIG. 1 is an external perspective view of a modified backpack used in the embodiment of a data-logging backpack, where the backpack portrayed is a typical backpack designed for school children.

FIG. 2 is a perspective view of one embodiment of the mechanical assembly. In the case of the embodiment where the mechanical assembly is being used in conjunction with a modified host backpack, the mechanical assembly partially resides inside the host backpack. For clarity, only the first top return spring is shown. The number of rods shown is only an example, and depends on the desired resolution.

FIG. 3 is a simplified side view of the invention, with the a surface of the host backpack removed to show the mechanical assembly inside. In this figure, an embodiment of the mechanical assembly is used in one embodiment of the invention, a data-logging backpack. The backpack assembly is connected to support equipment, showing the mechanical assembly, described above as FIG. 2, mounted inside a test backpack used in the invention.

FIG. 4 is a perspective view of the invention used in the embodiment of a backpack load-carriage measuring instrument, showing the placement of the vertical modification slot and mounting holes, and the sliding contact rods from the mechanical assembly.

FIG. 5 is a close-up perspective view of a section of the mechanical assembly used in the invention. The top-three rod assemblies are shown in detail, with their corresponding position sensors and optional support pieces.

FIG. 6 is a top view of a single rod section, detailing the placement possible sliding contact rod assembly components in one embodiment.

FIG. 7 is a simplified side-view abstract of a segment of the mechanical assembly's rods, their sensors, and contact disks, showing the translation of spine position into sensor position.

FIG. 8 is a circuit block-diagram of some of the invention's electronic and electrical sections, when a micro-controller is used in the embodiment of a data-logging assembly.

### DETAILED DESCRIPTION OF THE INVENTION

The following is a description of an embodiment of the mechanical assembly (claimed in claim 1), where the mechanical assembly is used to create a data-logging backpack assembly. This embodiment of a data-logging backpack assembly is claimed in claim 1, 2 and 3.

Further, the embodiment that follows assumes the user utilizes linear-travel variable resistors, as the contact rod sensors, in their design.

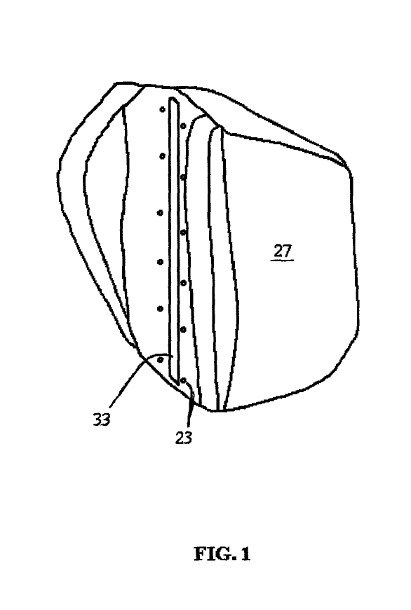

Referring to FIG. 1, the modified host backpack is shown to include ine parts 23, 27, and 33 as follows.

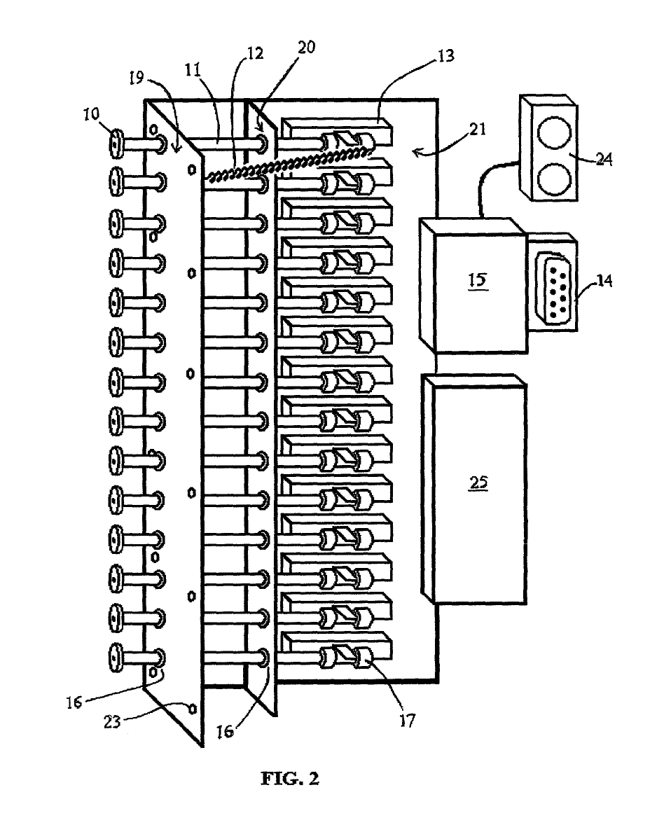

The modified host backpack 27 is the backpack chosen to house the mechanical assembly, described below, and shown in FIG. 2. The modified host backpack should be large enough to hold the mechanical assembly, which is described below.

When the invention is used in this embodiment, the invention effectively measures the load-carriage effects of the specific backpack (used as the host backpack 27) on the measured spine.

The host backpack 27 holds the mechanical assembly against the spine, so that the sliding contact rods 11 may perform properly, as previously described. In order to expose the sliding contact rods 11 to the spine, a vertical contact rod slot 33 may be cut in the backpack.

The modified host backpack 27 made of a material in which the contact rod slot 33, either a single slot or multiple slot sections, can be cut completely through the backpack surface nearest the spine.

The purpose of contact rod slot 33 is to allow physical contact between the sliding contact rods 11, and the spine under measurement, without interference from the backpack.

The contact rod slot 33 should have a width of at least the largest contact disk 10, if the optional contact disks 10 are used, so that when the sliding contact rods 11 are pressed in to their minimum protrusion, the contact disks 10 will be recessed into the contact rod slot 33.

The height of contact rod slot 33, if cut as a single slot, should be at least the distance between the centers of the first and last sliding contact rod 11 in the mechanical assembly. Alternately, several slots may be cut, such as individual holes for each sliding contact rod 11.

The mounting holes 23, or any connection devices used as the means for attaching the mechanical assembly to the modified backpack 27, should be distributed on the modified host backpack 27 in the same pattern as used in the mechanical assembly, to ensure that the two sections will align properly for the sliding contact rods 11 to protrude from the contact rod slot 33. The number of mounting holes 23 should be large enough that the mechanical assembly is firmly attached to the modified backpack.

The host backpack 27 to be used in conjunction with the mechanical assembly should be of the type where at least one surface normally touches the wearer's spine, or else, its position is fixed with respect to the wearer's spine. This includes virtually all two or more strap backpacks used today, where at least one point of the surface of the backpack, between the straps, is designed to support itself against the wearer's back.

FIG. 2, showing one embodiment of the mechanical assembly, designed to measure spine curvatures and movement when attached as described to a modified backpack (as in FIG. 1), is shown to include parts 10-17, and 19-26 described as follows.

Only one return spring 12 is shown for clarity; in the actual design, every sliding contact rod 11 may be connected to a return spring 12.

The placement of battery pack 25, electronic circuits 15, and computer connection cable 14 may be placed wherever they do not interfere with the operation of the invention. Suggested placements are indicated in the figure.

One embodiment of the bushing assembly, designed to hold the sliding contact rods 11, will be described:

The main support plate 21 is a base for front and rear bushing plates 19 and 20; all three are molded of a strong and rigid material, for example Aluminum. Front bushing plate 19 is mounted essentially perpendicular to the main support base 21. Its connecting edge may be flush with a leading edge of the main support base 21, to allow the mechanical assembly to sit perfectly flush against the modified backpack's rear surface. Rear bushing plate 20 is secured essentially perpendicular to the main support base 21, and essentially parallel to front bushing plate 19.

The primary purpose of front and rear bushing plates 19 and 20 is to hold the low-friction bushings, described below, for the sliding contact rods 11. Another purpose of front bushing is to allow a mounting location for the pressure and return spring 12. The third purpose of front bushing plate 19 is to allow a means to secure—via a series of mounting holes or other connection devices such as latches distributed throughout its surface—the mechanical assembly section of the invention to the modified backpack portion of the invention, using for example rivets or flathead machine screws or bolts.

In one embodiment, a reasonable distance between the two facing surfaces of bushing plates 19-20 is 15-20 times the sliding contact rod 11 diameter.

The bushings 16 is made of a material that exhibits low friction when rubbed against the material of a sliding contact rod 11, to prevent binding during movement. If the sliding contact rods 11 are made of Aluminum, for example, the bushings 16 could be made of Nylon or brass.

The bushings 16 may be arranged in a single column formation on both front and rear bushing plates 19-20. The columns of bushings 16 may be an equal distance from the main support base 21, at a maximum distance so that the end of the adjustment tabs of the variable resistors used as the contact rod sensors 13 (attached to the main support base 21), described below, reach the center of the bushings. Moreover, this distance can be arranged to be at least as large as a rod mounting cap's (17) outer radius, plus the height of the variable resistor base, to prevent the sliding contact rod movement from being inhibited by the collision of the sliding contact rod assemblies with the variable resistor housings.

The distance between the centers of neighboring bushings 16, or between the centers of simple holes if they are used instead, should be at least the diameter of the rod mounting caps 17, described below, to ensure that no binding will result between the neighboring rod mounting caps 17. If the width of the variable resistors used are larger than the outer diameter of the rod mounting caps 17, the minimum distance between bushings 16 can be made to be at least the width of the variable resistor.

Sliding contact rods 11 should be long enough so that, even with the rod mounting caps 17 in place, they can be displaced the entire desired effective measuring distance, typically on the order of 3-6 cm. This distance is entirely flexible, but larger distances should in most cases not be necessary. All sliding contact rods may have the same length for simplicity, however different lengths are allowed, as long as they are taken into account during calculations of the obtained data.

The contact disks 10 can be made of a rigid, non-flexible material such as Aluminum. The following is the design and purpose of the contact disks, in the case that rotating contact disks 10 are desired in the design.

The primary purpose of the rotating contact disks 10 is to increase the contact area. By increasing the contact area, the probability that measurements are less accurate, due to certain narrow sliding contact rods settling between vertebrae joints, and others on the peaks, is minimized. In effect, the distance each sliding contact rod 11 measures, is averaged, ensuring more consistency and reliability in measured results.

The secondary purpose of rotating contact disks 10 is to minimize rotation of the sliding contact rods 11, even though the contact point of each individual sliding contact rod is rotating. For example, the shirt of a test subject wearing this invention, due to lengthy walking in a measurement experiment, may wrinkle and begin to bunch up around the contact points without the rotating contact disks; thus leading to less accurate measurements. With the rotating contact disks, however, this behavior is minimized due to the circular uncoupling of the contact points to the sliding contact rods 11. In addition, rotation of the contact point is minimally translated to the rod mounting caps 17, thereby keeping the pressure and return spring 12 oriented correctly.

The outer diameter (o.d.) of the rotating contact disks should be limited so as to avoid inhibiting movement. Typically, the sliding contact rod 11 placements are determined, and then the rotating contact disk diameters are chosen. Rotating contact disks 10 may be mounted to the sliding contact rods 11 via a recessed screw.

The thickness of the rotating contact disks 10 depends on the material they are constructed of. They should be large enough that a securing hole and other normal loads on the disks 10, causes no weakening of the structural integrity of the disks 10 during normal use of this invention.

Rod mounting caps 17 may be employed at the end of the contact rods 11, to interface a sensor to a sliding contact rod 11.

The following is one embodiment of rod mounting cap 17, in the case that linear variable resistors are chosen as contact rod sensors 13.

The rod mounting caps 17 fit snugly onto the sliding contact rods 11. They can be secured with appropriate epoxy, or may be screwed on to the sliding contact rods 11, or secured by any means sufficient to withstand the force of the return spring 12.

The rod mounting caps 17 can be made of a material such as aluminum or nylon. The length of rod mounting caps 17 may have at least the width of the variable resistors adjustment tabs, if variable resistors are used as the contact rod sensors 13, to allow room for mounting the pressure and return spring (12), as described below.

The rod mounting caps 17 should have an o.d. large enough, so that even with a center portion of rod mounting cap 17 turned down to a secondary o.d., smaller than the original o.d. by about the thickness of a variable resistor adjustment tab, they remain structurally intact. In this manner, the adjustment tab may be seated into recessed section, without working its way out of the groove.

The center portion with the smaller o.d. should be at least as wide as the width of the adjustment tab of the chosen variable resistors, so that the adjustment tab seats neatly into the groove; however, the width of this section should not be so large as to allow too much backlash of the adjustment tab, when the rod mounting cap 17, connected to the sliding contact rod 11, is moved.

The purpose of the contact rod sensors 13 is to sense the sliding contact rod 11 positions, and so the sensors must have a designed sensing length of at least the desired sliding contact rod 11 sensing distance. In one embodiment, variable resistors are used as contact rod sensors 13. Variable resistors with a low-friction travel are preferred, in order to facilitate the use of low k pressure and return springs 12, which increase the accuracy of the resulting measurements by minimizing the interference and pressure of this invention on the test-subject's spine. Alternately, high-friction linear variable resistors can be carefully hand-modified to significantly lower their traveling resistance or friction, as was done with the working model of this invention.

If variable resistors are used as the contact rod sensors 13, the end-to-end resistance value is not critical, but a value larger than a few 1000 Ohms is preferable, since the value of the sensing resistor decides how much quescient current is drawn. Quescient current should be minimized to maximize battery life, but should not be so small that the low sensor output current causes unreliable readings from stray noise on the high-impedance micro-controller or Analog to Digital converter (ADC) inputs, if they are used.

The variable resistors, or alternately any sensors chosen as the contact rod sensors 13, may be mounted to the main support plate 21, with their sensing directions generally parallel to the sliding contact rods 11. Further, the variable resistors could be mounted so that the sliding adjustment tab of these resistors rest in the vicinity of their corresponding sliding contact rods, at such a position along main support base 21, that the adjustment tabs of the variable resistors fit neatly into the smaller o.d. center portion of the rod mounting cap 17 of that sliding contact rod. In addition, the variable resistors may be mounted a distance from the rear bushing plate 20, so that with the variable resistor snugly fitted inside the rod mounting cap 17, the sliding contact rod 11 can be displaced by its fill intended sensing distance.

The pressure and return spring 12 may be of the normally contracted type (a “pull” spring), and be made of a durable and non-fatiguing material such as spring steel. In another embodiment, the pressure and return spring 12 may be any device that naturally relaxes to a state in which it is a smaller length, such as an elastic strap.

One end of a pressure and return spring 12 can be mounted along the forward mounting plate 19, and as near as practical to the corresponding sliding contact bar 11 it will be acting on. The other end of the pressure and return spring 12 can be mounted to rod mounting cap 17 of this corresponding sliding contact bar 11. While connected in this fashion, the pressure and return spring 12 should preferably have a length such that, with the corresponding sliding contact rod 11 at its maximum intended protrusion distance, i.e. the rotating contact disk 10 of the latter rod is furthest from the forward bushing plate 19, the pressure and return spring 12 is still not at its relaxed original length. Similarly, with the sliding contact rod 11 at its minimum possible protrusion distance from the bushing assembly, the pressure and return spring 12 should preferably not be at its maximum stretch length.

The pressure and return spring should have a spring constant k large enough that the return force of the spring with its corresponding sliding contact rod 11 at its maximum protrusion, as described above, can overcome the static friction between the sliding contact rods 11 and bushings 16, plus the static friction in the contact rod sensor 13.

The diameter of the pressure and return spring 12 is not critical, however it should be reasonable, preferably such that the pressure and return spring 12 will not be in contact with the rear bushing plate 20.

The ends of the pressure and return spring 12 can be simply terminated to the bushing assembly, the rod mounting cap 17, or the sliding contact rod 11. In one embodiment, the wire at the ends of the spring are formed into small loops, and hooked through holes on the desired termination point.

An on-board battery pack 25 powers the contact rod sensors 13 and the circuitry 15, which may include the recording device. In a preferred embodiment, the recording device is a micro-controller with necessary components, described below. Alternatively, a (preferably voltage regulated) external tethered power supply 32 may be used in place of the battery pack 25 for stationary tests, where a power umbilical would not need to be long, for example tests conducted on a treadmill.

If a tethered power-supply is not used, the battery pack 25 may be rechargeable to optionally allow easy charging without removal from the backpack. Because the TTL logic used in most circuitry requires stable voltage, a regulator may be used with the battery pack. The battery pack should be at least 7.5V when using a standard inefficient regulator such as the lm7805, and at least 6V when using a Low Drop-Out regulator such as the lm2941, considering that most logic requires 5V. The recommended approach is to use a switching regulator, such as the lm12595 or Power Trends PT75HCT05, which provides a low-ripple output, has high efficiencies, and requires only 5.5V to operate within specifications.

A voltage regulator is one means to achieve the stable output the microcontroller and various circuit elements require. The 0-5V output of the contact rod sensor 13, in the embodiment of a linear variable resistor, corresponds linearly to the sliding contact rod 11 position, if the contact rod sensor 13 used has a linear output. This output then indicates the position of the spine portion the said sliding contact rod 11 is contacting. When using a sensor such as an LVDT, the input is an alternating voltage source (AC), which may be provided separately from the DC 5V circuitry voltage. The output of an LVDT is also AC, and so must be rectified in order to not damage the ADC inputs via the negative voltage swings. More circuitry is necessary to condition the LVDT differential output, otherwise the LVDT outputs will be influenced by temperature and humidity swings, leading to less accurate results

For AC output sensors, such as LVDTs, fast-sampling ADCs (with sampling rates preferably at least twice as high as the AC voltage frequency) may be used for accurate measurements of the peak voltage values, which are what correspond to the sensor positions.

The additional circuit complexity of a design using LVDTs, despite the more robust and simpler mechanical design, may make the simpler variable resistor approach more attractive.

User input buttons 24 may be added as inputs to micro-controller 28, to allow various setting various program parameters conveniently, for example fixing data sampling periods, timers to start data recording, downloading onboard data to the host computer 29, or choosing which stored data-collection program to execute.

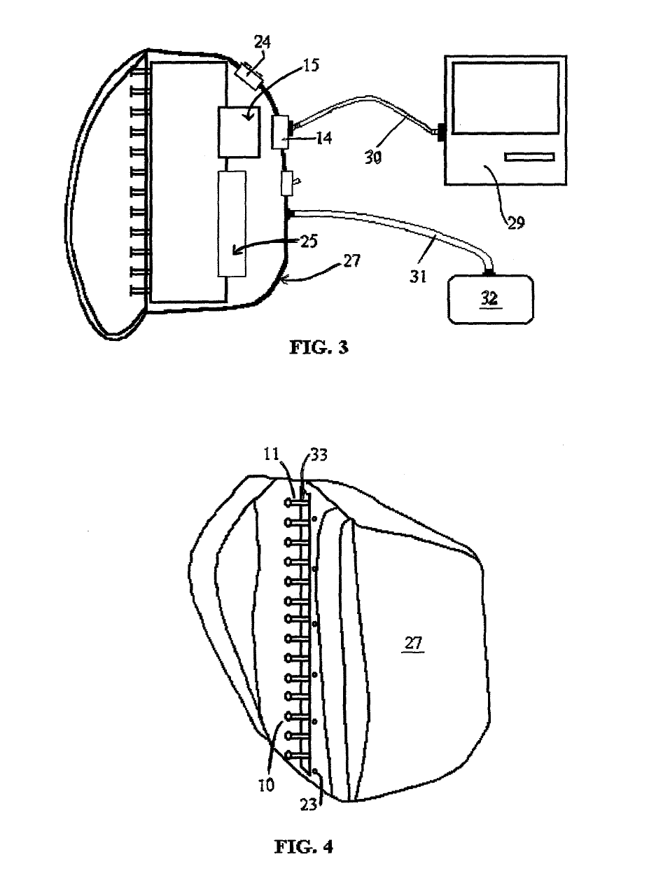

Referring to FIG. 3, a connection diagram of one embodiment of the invention's electronic systems to the host computer, is shown to include parts 14-15, 24, 25, 27 and 29-32 as follows.

In an embodiment of the invention being used as a data-logging backpack assembly, where the recording device is a micro-controller assembly:

To physically connect the host computer with the micro-controller 28, a computer cable connector 14 may be used. Any standard computer cable 30 with a suitable adaptor can be used to transmit data and programs to the microcontroller 28 (a section of the circuitry 15) from the host computer 29, and from the host computer 29 to the micro-controller 15. The host computer 29 should have the ability to transmit and receive serial data, and capture the serial data, sent from the micro-controller 15, to a file; such computers are commonly, cheaply, and easily obtained. The data can then be loaded and saved by the host computer, and analyzed at any time.

One or more of the user input buttons 24 may be used to signal to the micro-controller 28, when to transmit data to the host computer 29, or which recording program the micro-controller should run. The host computer 29 is shown connected to this embodiment of the invention, although it does not need to be connected during remote tests. The micro-controller 28 can transmit the recorded data to the host computer 29 automatically, or display the data to a liquid crystal display (LCD) attached to the backpack.

In one embodiment, the modified host backpack 27 holds the said components. The modified host backpack is shown as a reference to suggested placements of the components, described above, in this embodiment. The backpack's electronic systems may be powered by a battery pack 25, or an external power supply 32, connected through a power umbilical 31; both options are shown in the figure.

Referring to FIG. 4, a perspective view of one embodiment of the modified host backpack with the mechanical assembly mounted inside the modified host backpack, is shown labeled with parts 10, 11, 23, 27, and 33, as follows.

The sliding contact rods 11 emerge through the contact rod slot 33, showing the configuration before the compression of any of the sliding contact rods 11. In this embodiment, the mechanical assembly is securely mounted onto the modified host backpack 27, by using mounting holes 23.

The contact rod disks 10 are shown with the sliding contact rods 11, since these may be the only components that emerge from inside the modified host backpack 27.

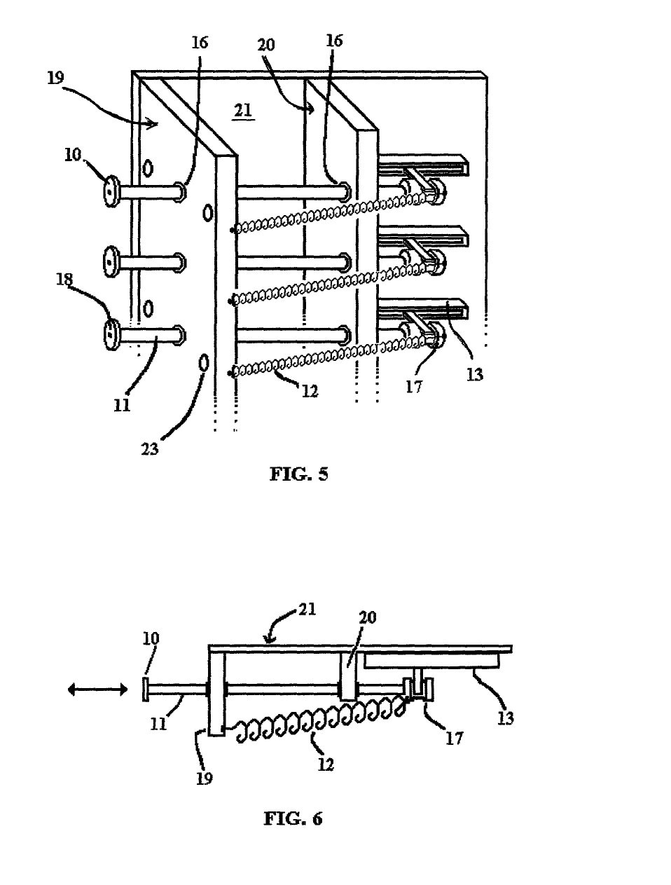

FIG. 5 is a close-up and more detailed depiction of one embodiment of the sliding contact rod assemblies in the mechanical assembly section of the invention, with the placements shown here in a higher detail than in FIG. 2, and is shown to include the parts 10-13, 16-21, and 23 as follows:

The section shown here is the top portion of the mechanical assembly. Other components of the invention are left off for clarity and simplicity. The dotted lines are included only to indicate where the remainder of this section of the mechanical assembly is located.

The rod mounting cap 17 serves to connect the adjustment tab of the variable resistor, which is used as the contact rod sensor 13, to the sliding contact rod 11, and shows its placement with greater detail than possible with a full view of the mechanical assembly.

Parts 10-13, 16-21, and 23 are included to show suggested placements of various components, of the mechanical assembly in FIG. 2, in greater detail.

FIG. 6 is a top-view of one embodiment of a single sliding contact rod assembly, as shown in FIGS. 2 and 5, including the parts 10-13, 17, and 19-21. This view portrays the layout of the pressure and return spring 12 and contact rod sensor 13 (in this embodiment a linear variable resistor) relative to the sliding contact rod 11 and rod mounting cap 17.

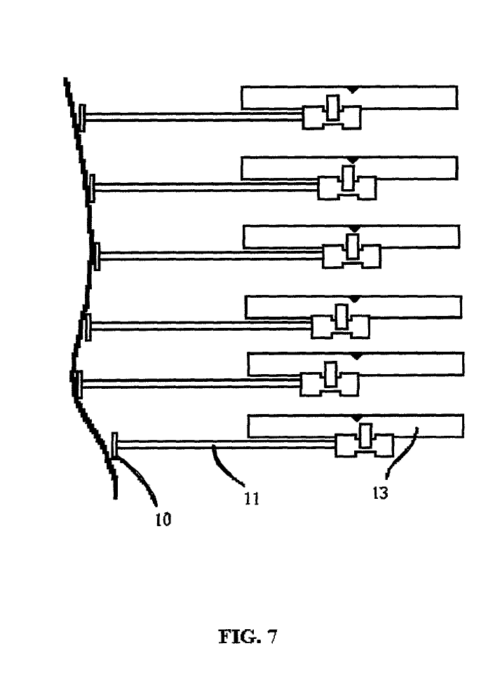

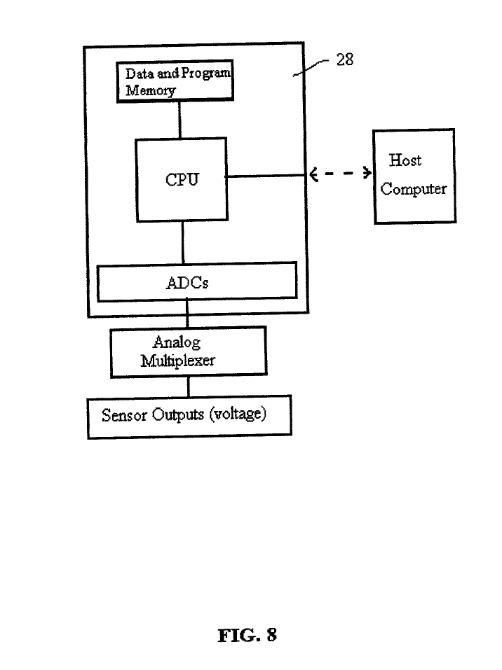

FIG. 7, including parts 10, 11, and 13, is a side-view of several sliding contact rod assembly sections of one embodiment of the mechanical assembly, portraying how sensor displacement-from a reference point (marked as an inverted triangle on the contact rod sensors13)—is dependent on spine curvature. The solid curved line on the extreme left of this figure is a representation of a side view of a curved spine being measured.

The displacement from an common arbitrarily chosen reference point for each individual sensor results in an immediate correspondence of sensor output to spine position, due to the direct translation of spine segments to their corresponding contact rod sensor 13 positions.

In the case that one point of the back section of the backpack is always touching the wearer's back, at least one sliding contact rod II will always be at its minimum extension (maximum depression), and these will act as the reference for all additional sliding contact rods 11. Note that these reference rods may change freely during a trial; due to the inherent geometric nature of the relative measurements of this invention, results are not affected.

Contact disks 10 are properly extended until they are properly inhibited by the spine being measured; the elastic member used to exert pressure on the sliding contact rods 11 exerts a force which points generally towards the spine being measured.

Referring to FIG. 8, one embodiment of the composition of microcontroller 28 chosen as the recording device, and various support circuitry components, is shown to include part 28 described as follows.

The micro-controller 28 used should have enough data memory (preferably EEPROM or NVRAM) to store the desired data amount. In addition, the program memory should be large enough to store a simple program, for example, ADC collection schemes with on-board data filtering, multiple data collection modes, or however else the user desires to record and store the data. In one embodiment, a the working model of the invention, about 5 k of program memory was sufficient. The program memory can be any type of memory desired (such as EPROM, EEPROM, RAM, ROM, NVRAM). For low-level languages, less may be necessary. The program memory can be physically in the same memory block as the data memory.

To read the variable resistor position, Analog to Digital converters (ADC's) that can read at least GND to Vcc, should be either externally connected to the micro-controller 28, or be integrated into the micro-controller 28. In addition, the ability to communicate serially with a host computer is important, in order to download and upload data and programs. This allows the easiest analysis of collected data.

Micro-controllers that fit the requirements above are commonly and inexpensively available. Examples of micro-controllers meeting all requirements for data and program storage, serial communications, and integrated ADCs include the high-end of Microchip's PicMicro series, Motorola's MM68HC12 series, or Netmedia's Basic Express 24 (BX-24). The latter was used in the working model. For other micro-controllers with no internal ADCs, ADCs such as the common AD0831 are available.

Depending on the measurement resolution desired of this invention, there can be a desire to input more contact rod sensor 13 outputs to the microcontroller 28 than exists ADC input pins on the said micro-controller. In this case, any common analog multiplexer chip may be utilized, which can expand the number of available ADC pins. As an example, one such chip includes the CD4051 8 to 1 analog multiplexer chip, several of which were used in the working model. When using this, and other CMOS chips, with the invention, care should be taken when insulating, grounding, and mounting the circuitry, so that static build up, both from electrically charged humans or the sliding contact rod 11 movement, does not discharge onto the circuitry, ruining the sensitive CMOS chips.

### LIST OF REFERENCE NUMERALS

10 Contact disk

11 Sliding contact rod

12 Pressure and return spring

13 Contact rod sensor

14 Computer cable connector

15 Electronics circuits

16 Bushings

17 Rod mounting cap

19 Forward bushing plate

20 Rear bushing plate

21 Main support plate

23 Mounting holes

24 User input buttons

25 Battery pack

27 Modified backpack

28 Micro-controller

29 Host computer

30 Computer cable

31 Power umbilical

32 External power supply

33 Contact rod slot

What is claimed as my invention is:

1) A method for measuring spine curvatures and spine contours, comprising:
(a) deploying movable rods, generally perpendicular to the spine being measured, capable of being displaced primarily in a direction parallel to their length, by a distance depending on the position of the spine segment being measured, contained in a portable apparatus,
(b) sensing the displacement of said movable rods with linear-sensing sensors attached to said movable rods.

2) Method of claim (1), wherein recording said displacements of said movable rods is done by a recorder carried by the test subject whose spine curvature is being measured.

3) The method of claim (1), wherein said apparatus is attached to a host backpack carried by said test subject, with said host backpack being modified in a manner such that said apparatus measures the load carriage effects of said host backpack on the wearer's spine.

4) An apparatus consisting of a portable mechanical assembly for measuring spine position and contours, comprising:
(a) sliding contact rods, generally perpendicular to the spine being measured, capable of being displaced primarily in a direction parallel to their length, by a distance depending on the position of the spine segment being measured,
(b) a rigid bushing assembly which contains devices that allows each said sliding contact rod to independently move on an axis generally parallel to its length, but not move significantly in any other direction,
(c) elastic members such as return springs, mounted to each said sliding contact rod, with the purpose to exert pressure on said sliding contact rod, with this pressure being primarily directed parallel to the length and travel of said sliding contact rod, arranged in such a manner that the more relaxed state of said return springs occurs when said sliding contact rod extends out and away from the bushing assembly,
(d) linear-sensing sensors attached to said sliding contact rods, sensing the displacements of said sliding contact rods.

5) The apparatus of claim (4), wherein said mechanical assembly is employed as a spine-measuring instrument on a freely moving test subject, said sliding contact rod position data being sensed and recorded by a recording device carried by said test subject, allowing for the downloading of this data later to a host computer.

6) The apparatus of claim (4), wherein a backpack is modified by means of altering portions of the backpack to allow said sliding contact rods, originating from the mechanical assembly, to extend from the inside of the backpack to their maximum designed travel, with minimal function between the backpack and said sliding contact rods.
